(19) United States
(12) Patent Application Publication
(10) Pub. No.: US 2026/0145144 A1
(43) Pub. Date: May 28, 2026

(54) Stable Carbonic Acid and Method of Making

(71) Applicants: Shane Sauceman, Andersonville, TN (US); Donald Jeffrey, St. Andrews (CA); Lewis Malone, McEwen, TN (US)

(72) Inventors: Shane Sauceman, Andersonville, TN (US); Donald Jeffrey, St. Andrews (CA); Lewis Malone, McEwen, TN (US)

(21) Appl. No.: 19/391,540

(22) Filed: Nov. 17, 2025

Related U.S. Application Data
(60) Provisional application No. 63/724,564, filed on Nov. 25, 2024.

(51) Int. Cl. B01F 23/237; A61K 9/08; A61K 47/02; A61K 47/10; A61K 47/36; A61K 47/38; B01F 23/231; B01F 35/22; B01F 35/221; B01F 101/14

(52) U.S. Cl. CPC B01F 23/23762; A61K 9/08; A61K 47/02; A61K 47/10; A61K 47/36; A61K 47/38; B01F 23/231244; B01F 23/237612; B01F 23/237621; B01F 23/23764; B01F 35/2202; B01F 35/2211; B01F 2101/14; B01F 2215/044

(57) ABSTRACT

A carbonic acid solution comprises an aqueous infusion of CO₂ having a pH≤5 and characterized in that the pH is substantially stable for at least 30 days in an open container at ambient temperature and normal atmospheric pressure. The carbonic acid solution may be further characterized in that the scattered intensity of a polarized laser beam passing therethrough is at least 2 times greater in the direction parallel to said polarization than the scattered intensity in the direction normal to the plane of polarization. The solution may be further infused with other gases and may be mixed with gel-forming materials and other additives. A related method for making the carbonic acid is also disclosed.

### CROSS REFERENCE TO RELATED APPLICATIONS

This application claims the benefit of U.S. Provisional Patent Application 63/724,564 filed on Nov. 25, 2024, entitled, “Gas Infusion Apparatus and Method of Making”. This application is related to U.S. patent application Ser. No. ______ filed on even date herewith, entitled, “Gas Infusion Apparatus and Method of Making.” This application is also related to U.S. patent application Ser. No. ______ filed on even date herewith, entitled, “Monitoring Device for Stable Carbonic Acid Solutions.” The entire contents of each of the aforementioned disclosures are incorporated herein by reference.

### BACKGROUND OF THE INVENTION

### Field of the Invention

The invention pertains to apparatus and methods for infusing gases into fluids, and more particularly, to a gas infusion apparatus and process that yields a carbonic acid solution characterized by a high degree of stability and distinctive optical properties.

### Description of Related Art

There are many applications, in diverse fields, in which a gas exchange membrane is used to infuse a fluid (typically water or some aqueous solution) with a selected gas (typically oxygen or carbon dioxide). The usefulness of oxygenated water is well known in such fields as commercial aquaria and fish hatcheries, hydroponic gardening systems, and wound care. Carbon dioxide may be infused into water to produce carbonic acid for various purposes. Many other combinations of liquids and gases are also known to be suitable for gas infusion processes.

The basic features of a gas infusion system include a vessel with a water inlet and outlet and a gas inlet. Water flows through the vessel and around a membrane contactor defining a gas-filled space separated from the water-filled space by the membrane, which is hydrophobic and gas permeable. Conventionally, the membrane is constructed as a bundle of very small tubes connected to a gas collection chamber pressurized with the selected gas via the gas inlet. The tubes are typically polyethylene, polypropylene, or other hydrophobic polymer with an outer diameter around 500 μm and inner diameter around 350 μm and a porosity of around 75% [see, e.g., U.S. Pat. No. 6,209,855 to Glassford]. The tubes are flexible and mechanically fragile and are connected to the gas collection chamber by first potting the tube ends in a thermosetting or photocuring resin to form a rigid disk. The bottom surface of the disk is then machined away to expose the ends of the tubes so that gas may enter them from the gas collection chamber. This process is tedious and typically some or many of the tube ends are deformed or crushed so that an unknown number of the tubes receive no gas from the chamber and therefore those tubes do not contribute to gas infusion. Furthermore the tubes are very thin compared to their active length (often up to 30 cm long) making them mechanically fragile, so that excessive fluid turbulence can be damaging to the apparatus. Another shortcoming of these systems is that if the module is left submerged in water without maintaining internal gas pressure, the individual fibers will become internally filled with water, and once in that condition they must be thoroughly dried before that can be used again.

### Objects and Advantages

Objects of the present invention include the following: providing a gas exchange membrane that is physically robust; providing a gas infusion apparatus that is manufacturable; providing a gas infusion module that incorporates integral features to induce fluid turbulence; providing a method for constructing a monolithic gas infusion module constructed from a single polymeric material; providing a stable carbonic acid solution that does not immediately deteriorate when exposed to ambient atmospheric conditions; providing a sensing apparatus to evaluate the quality of stable carbonic acid solution at the point of production and/or use; and providing useful products and processes enabled by a stable carbonic acid solution.

### BRIEF SUMMARY OF THE INVENTION

According to one aspect of the invention, a gas exchange membrane comprises: a rigid, monolithic polymer body defining an internal cavity and an external surface; a gas inlet integral to the polymer body so that pressurized gas may be introduced into the interior cavity and maintained at a selected pressure therein; and wherein, the polymer body is sufficiently hydrophobic so that when the body is immersed in water, the gas will diffuse from the internal cavity to the external surface and enter solution in the water.

According to another aspect of the invention, a gas infusion apparatus comprises:
a rigid, monolithic polymer body defining an internal cavity and an external surface;
a gas inlet integral to the polymer body so that pressurized gas may be introduced into the internal cavity and maintained at a selected pressure therein;
a rigid housing defining a fluid chamber surrounding the polymer body, the housing having a water inlet, and a water outlet, and wherein the gas inlet passes through the housing wall from the polymer body to an external gas connection; and wherein,
the polymer body is sufficiently hydrophobic so that when water is passed through the fluid chamber, the gas will diffuse from the internal cavity to the external surface of the polymer body and enter solution in the water as it passes through the fluid chamber.

According to another aspect of the invention, a method of making a gas infusion apparatus comprises the steps of:
a) creating a digital solid model defining:
   an outer shell having a water inlet, a water outlet, and a gas inlet,
   an inner hollow body integrally connected to the gas inlet so that a selected gas can be maintained at a selected pressure inside the inner hollow body while the outer shell is filled with water;
b) uploading the digital solid model to an additive manufacturing system;
c) loading the additive manufacturing system with a feedstock comprising a selected thermoplastic hydrophobic polymer;
d) building a monolithic polymer body wherein the inner hollow body is sufficiently gas permeable so that when gas is supplied via the gas inlet and water is passed through the outer shell, gas passes through the surface of the hollow body and into the water.

According to another aspect of the invention, a bottling line for beverages comprises a gas infusion module for adding selected gases to the beverage during the bottling process, the gas infusion module comprising:
a rigid, monolithic polymer body defining an internal cavity and an external surface;
a gas inlet integral to the polymer body so that pressurized gas may be introduced into the internal cavity and maintained at a selected pressure therein;
a rigid housing defining a fluid chamber surrounding the polymer body, the housing having a beverage inlet, and a beverage outlet, and wherein the gas inlet passes through the housing wall from the polymer body to an external gas connection; and wherein,
the polymer body is sufficiently hydrophobic so that when the beverage is passed through the fluid chamber, the gas will diffuse from the internal cavity to the external surface of the polymer body and enter solution in the beverage as it passes through the fluid chamber.

According to another aspect of the invention, a clean-in-place system for use in stainless steel process equipment includes a passivation system to provide oxygen-supersaturated water for passivating the stainless steel, wherein the passivation system comprises:
a rigid, monolithic polymer body defining an internal cavity and an external surface;
a gas inlet integral to the polymer body so that pressurized oxygen-containing gas may be introduced into the internal cavity and maintained at a selected pressure therein;
a rigid housing defining a fluid chamber surrounding the polymer body, the housing having a water inlet, and a water outlet, and wherein the gas inlet passes through the housing wall from the polymer body to an external gas connection; and wherein,
the polymer body is sufficiently hydrophobic so that when water is passed through the fluid chamber, the oxygen-containing gas will diffuse from the internal cavity to the external surface of the polymer body and enter solution in the water as it passes through the fluid chamber.

According to another aspect of the invention, a carbonic acid solution comprises:
an aqueous solution of CO₂ having a pH in the range of 4.0 to 5.0 and characterized in that said pH is substantially stable for at least 30 days when said solution is open to the atmosphere at ambient temperature.

According to another aspect of the invention, a carbonic acid solution comprises:
an aqueous solution of CO₂ having a pH in the range of 4.0 to 5.0 and characterized in that the ratio of scattered intensity of a plane polarized laser beam passing through said solution is at least two times greater in the direction of said polarization than the scattered intensity normal to the direction of said polarization.

According to another aspect of the invention, a monitoring device for a carbonic acid solution comprises:
a container for holding a selected volume of said solution;
a source of a plane polarized laser beam propagating longitudinally through said solution;
two photodetectors arranged to measure the intensity of light from said propagating laser beam scattered in two respective directions normal to the beam path, wherein the first of said two directions is parallel to the direction of polarization and the second of said two directions forms a nonzero angle with the direction of polarization.

According to another aspect of the invention, an infusion apparatus for producing stable carbonic acid solution comprises:
a gas infusion module comprising a monolithic hydrophobic gas-exchange membrane further comprising a water inlet, a CO₂ inlet, and a water outlet;
a source of pressurized water from a selected supply into the water inlet;
a source of CO₂ gas;
a monitoring device downstream from the water outlet to monitor the stable carbonic acid solution; and,
a control system to adjust the flow of inlet water based on feedback from the monitoring device.

According to another aspect of the invention, a hydrometallurgical process comprises exposing a crushed ore material to a leaching solution for a sufficient time to solubilize at least one metallic species from the ore,
wherein the leaching solution comprises stable carbonic acid solution.

According to another aspect of the invention, an in situ leaching process comprises the steps of:
a) drilling an injection well and at least one extraction well into a permeable layer of a selected metal ore;
b) forming a stable carbonic acid solution by infusing CO₂ into water using a gas exchange membrane module;
c) forming a leaching solution comprising the stable carbonic acid solution;
d) injecting the leaching solution into the injection well; and,
e) extracting a solution of said selected metal from the extraction well(s) in order to recover the selected metal.

According to another aspect of the invention, a skin treatment method comprises exposing the skin to a topical treatment for a selected time, wherein the topical treatment comprises exposure to a composition comprising stable carbonic acid.

According to another aspect of the invention, a method for treating a wastewater pond containing hazardous materials in a sediment comprises:
a) providing a gas infusion apparatus comprising a source of CO₂ and an infusion module to infuse the CO₂ into a process water stream to create a stable carbonic acid solution;
b) providing a fluid handling system including a pump, a water inlet line, and a water outlet line so that water may be extracted from the pond, passed through the infusion module, and re-injected into the pond; and,
c) operating the gas infusion apparatus for a sufficient time to effect one of the following chemical reactions:
   reacting with soluble metal species to form carbonate minerals to cement sediments into a less-soluble form and thereby reduce the concentration of hazardous metals in the pond water;
   reacting with solid metal species in the sediment to form soluble complexes that may be removed from the pond for recovery;
   solubilizing adsorbed metal ions in the sediment to form soluble complexes that may be removed from the pond for recovery.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore non-limiting embodiments illustrated in the drawing figures, wherein like numerals (if they occur in more than one view) designate the same elements. The features in the drawings are not necessarily drawn to scale.

FIG. 1A-B is a schematic diagram of a conventional gas exchange module in accordance with the PRIOR ART. FIG. 1A presents an exterior view and FIG. 1B presents a cut section along A-A.

FIG. 2A-C is a schematic diagram of a gas exchange membrane made by fused deposition modeling (FDM) in accordance with some aspects of the invention. FIG. 2A presents an oblique view, FIG. 2B presents an exterior elevation view, and FIG. 2C presents a cross section along A-A.

FIG. 3A-B is a schematic diagram of a gas infusion device in accordance with some aspects of the invention. FIG. 3A presents an external view and FIG. 3B presents a cross section. The device comprises an outer shell and an inner gas exchange membrane, all formed as one monolithic structure by an additive manufacturing process.

FIG. 4A-D is a schematic diagram of a gas infusion device in accordance with some aspects of the invention. FIG. 4A presents an external oblique view and FIG. 4B presents a side view. FIGS. 4C and 4D present cross sections along A-A and B-B respectively. The device comprises an outer shell and an inner gas exchange membrane, all formed as one monolithic structure by an additive manufacturing process.

FIG. 5A-B presents a side-by-side comparison of a conventional infusion module, FIG. 5A, and a module of the invention having equivalent infusion capacity, FIG. 5B.

FIG. 6A-C illustrates schematically an infusion module in which water channels run in a helical path from one end of the module to the other, and all the channels are surrounded by a gas plenum. FIG. 6A is a top view, FIG. 6B is a side view, and FIG. 6C is a section view along A-A.

FIG. 7 presents a plot of pH versus time, showing the stability of infused carbonic acid made according to some aspects of the invention.

FIG. 8A-B presents images of a laser beam propagating in a carbonic acid solution at pH=4.08. FIG. 8A is viewed in the direction of polarization, and FIG. 8B is viewed normal to the polarization.

FIG. 9A-B presents images of a laser beam propagating in distilled water solution at pH=7. FIG. 9A is viewed in the direction of polarization, and FIG. 9B is viewed normal to the polarization.

FIG. 10A-B presents images of a laser beam propagating in rain water at pH=6.49. FIG. 10A is viewed in the direction of polarization, and FIG. 10B is viewed normal to the polarization.

FIG. 11A-B is a schematic diagram of a sensor module in accordance with one aspect of the invention. FIG. 11A is an elevation view in cross section and FIG. 11B is a cross section through A-A, and the arrow indicates the direction of polarization of the laser beam.

FIG. 12 presents an exploded view of a sensor for static laser scattering measurements on a fluid sample.

FIG. 13 presents a cross sectional view of a sensor for continuous laser scattering measurements on a fluid stream.

### DETAILED DESCRIPTION OF THE INVENTION

In its most general form, the invention involves a gas-exchange device constructed of a thermoplastic polymer and forming a hollow body of a selected wall thickness, with an integral gas inlet so that the interior space or cavity may be kept filled with a selected gas at a selected pressure. The physical dimensions and gas permeability of the body are such that the pressurized body can be immersed in water and the gas will pass through the wall and infuse the surrounding water. The device is preferably made as a single, monolithic structure by additive manufacturing, i.e., 3D printing.

The invention fundamentally differs from conventional gas infusion devices that rely on long, thin, hollow polymer fibers, the ends of which are potted in a tube sheet of epoxy or similar cross-linked resin. Some clear advantages of the invention over the conventional approach include: easy manufacturing and customization; high mechanical strength and integrity; damage resistance; resistance to internal “flooding” when pressurized gas is absent; and improved efficiency from eliminating damaged or nonfunctional fibers.

In the examples that follow, when read in conjunction with the drawings, various aspects and advantages of the invention will become clear.

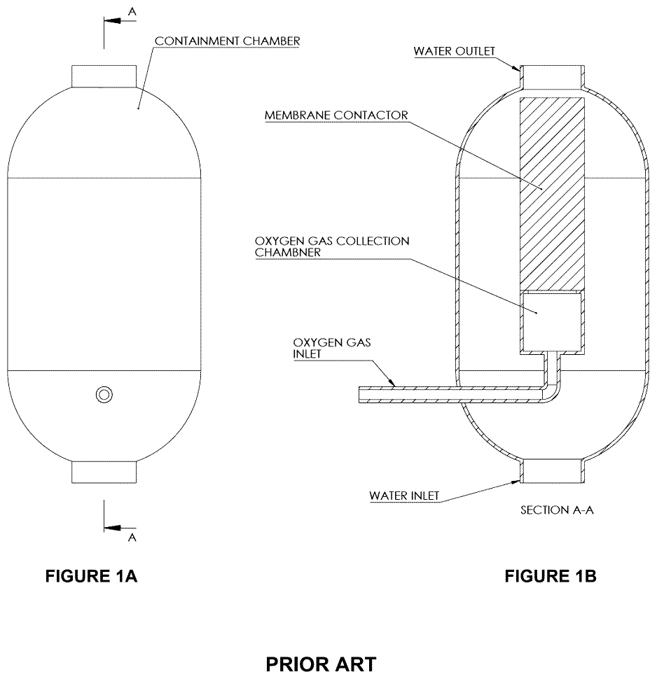

FIG. 1 presents schematically the external and internal configuration of a gas exchange module in accordance with the Prior Art. An external chamber, which may be metal or a structural polymer, has water inlet and outlet ports so that water to be treated passes through the space surrounding the membrane contactor. Gas is introduced through the gas inlet and fills a collection chamber or plenum. Thin hollow fibers of a gas-permeable hydrophobic polymer are potted in a rigid plate of thermoset polymer, which is then machined to form the upper surface of the collection chamber. Gas therefore fills the hollow fibers, diffusing through the fiber walls and into the fluid as it passes through the annular space in the external chamber. In one example, the fibers are polyethylene with an outer diameter around 500 μm and inner diameter around 350 μm and a porosity of around 75%.

The individual fibers are flexible and mechanically weak. Further, many are damaged by the potting and subsequent machining process so their ends are partly or completely closed off and no gas can enter. These nonfunctioning fibers therefore contribute nothing to the infusion process and overall efficiency thereby suffers.

The invention relies on additive manufacturing to create a completely monolithic gas exchange module, eliminating the fragile fibers and the cumbersome assembly process. The resulting structure, surprisingly, has demonstrated very efficient gas transfer.

### Example

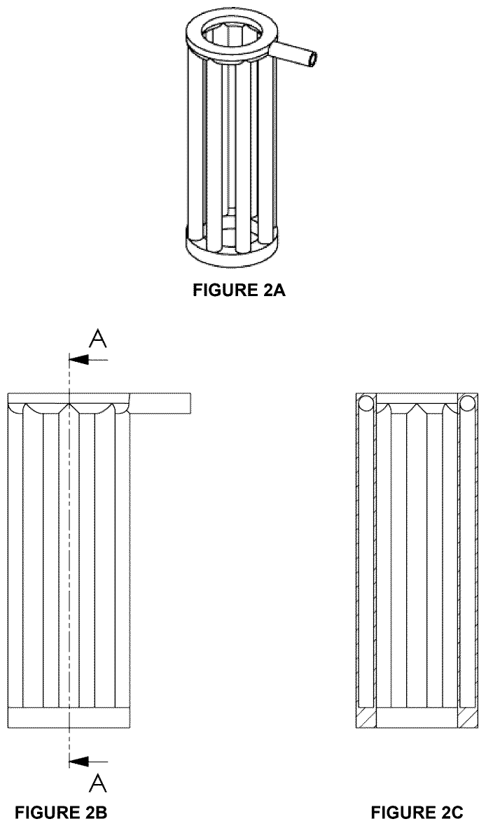

FIG. 2 shows a monolithic gas exchange membrane having an integral gas inlet and produced entirely by 3D printing. In this example, the feed material was polylactic acid (PLA). The build was done by fused deposition modeling (FDM) on an Ender-3 S1 3D Printer (Shenzhen Creality 3D Technology Co., Ltd., Shenzhen, China). Print speed was set using manufacturer's recommendations (layer height of 0.28 mm and print speed of 50 mm/s).
The membrane unit has an overall height of 10 cm and outer diameter of 2 cm. The gas collector has an internal volume of about 16 cm³. Individual tubes are 9.5 cm long, with an o.d. of 6.35 mm and an i.d. of 4.74 mm.

### Example

The membrane in the previous example was tested by connecting the gas inlet to a flexible tube and supplying oxygen regulated to a nominal pressure of 20 psi and flow rate of 2 L/min. The membrane was immersed in water and bubbles immediately began to appear. Occasional streams of larger bubbles were interpreted to be macroscopic oxygen leaks from small fissures or other minor defects in the build process. A more uniform distribution of smaller bubbles over the surface of the membrane was interpreted to be from the nucleation of nitrogen bubbles as the oxygen entering solution in the water displaced the dissolved nitrogen.
The membrane was placed into an open container filled with 2 cups of tap water with initial dissolved oxygen (DO) level of 83.7% of saturation. After 5 min the DO increased from 83.7 to 226.7% of saturation and the nitrogen decreased from 105% to 80.5% of saturation.
The membrane was structurally robust and was easily handled; the flexible gas feed tube was pushed onto the inlet and pulled off repeatedly without any damage to the body.

An immediately surprising aspect of the invention is that the oxygen infusion process was rapid despite the fact that the wall thickness of the 3D printed tube was many times greater than the wall thickness of the hollow fibers used conventionally (˜1 mm versus ˜50 to 90 μm).

It will be appreciated that the 3D printing process can easily fabricate very complex structures with internal cavities and passages. Applicant therefore designed a completely monolithic, self-contained gas infusion module in which an outer shell has a gas inlet and water inlet and outlet, and inside of this shell is a complete gas exchange membrane structure with its gas inlet forming a continuous extension of the gas inlet that penetrates the outer shell.

### Example

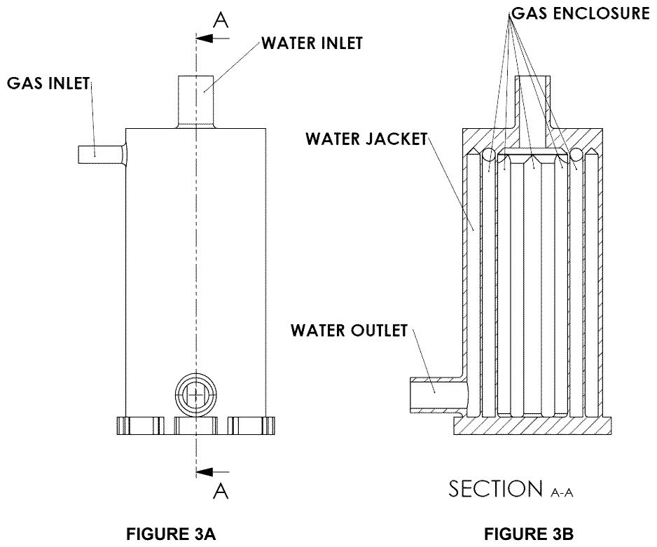

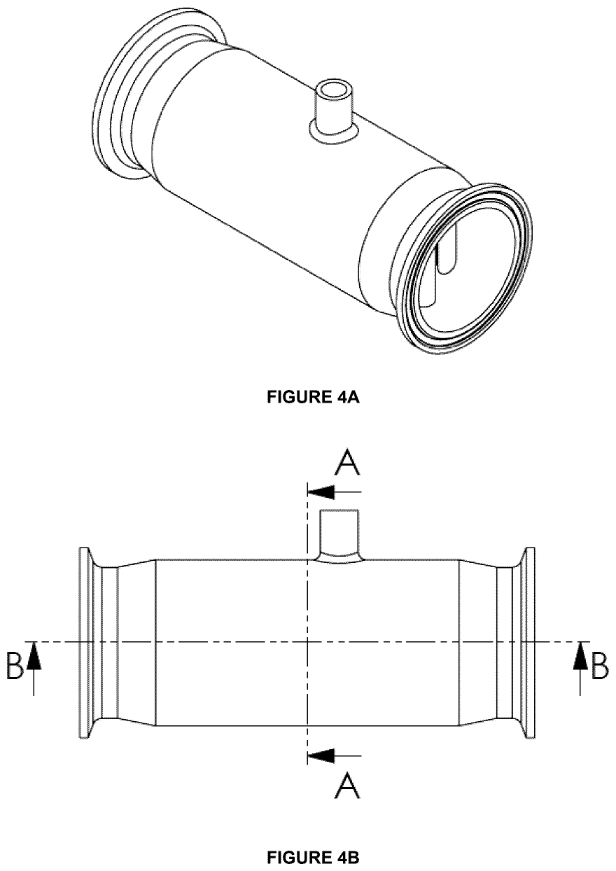

FIG. 3 presents schematically, in elevation view and cross section, a 3D model of a completely monolithic, self-contained gas infusion module having water inlet and outlet and gas inlet, ready to be connected directly to supply gas-infused water to any desired process stream. This module was tested by connecting water hoses to the inlet and outlet, with a recirculating pump on the inlet side. Both hoses were placed into a bucket containing about 5 gal of tap water, and the recirculating pump was operated at the rate of 5 gal/min so that the entire contents of the water bucket passed through the module about once per minute.
Starting with fresh tap water, oxygen was introduced into the module at the rate of about 2 L/min. The dissolved oxygen level in the water rapidly increased and within 4 min it had reached a level of about 300% of the normal saturation level in water at ambient temperature.
Starting again with fresh tap water, carbon dioxide was introduced into the module at the rate of about 2 L/min. The starting pH of the water was 7.0 and after about 4 min the pH was 4.9, indicating rapid infusion of the CO₂ and corresponding formation of carbonic acid.

It will be appreciated that the small device shown in FIG. 3 is a simple structure having the necessary features to carry out the inventive process. However, the skilled artisan will easily understand that the invention may be further modified in many ways for added convenience, performance or functionality. For example, the outer shell may be configured to include a removable panel or door that can be opened to allow cleaning of the tubes if the device is operating in water with a high level of impurities (e.g., a fish tank). Such features as hinges, O-ring grooves, etc., may be conveniently added during the design stage.

### Example

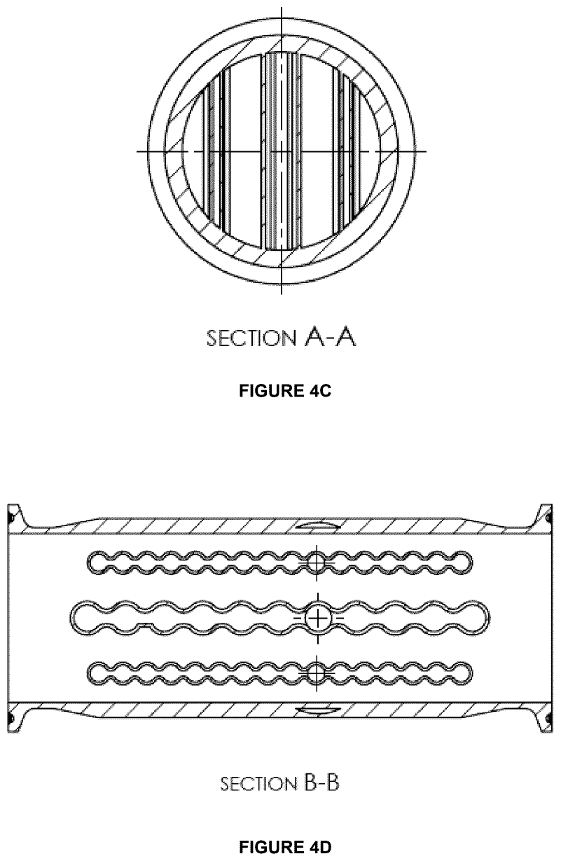

FIG. 4 presents schematically another configuration of the inventive module. In this example, there is an annular gas plenum in the wall of the pipe, and connected to this are individual tubes running transverse to the direction of water flowing through the pipe. These tubes may be arranged in groups at selected locations along the length of the pipe, and it will be appreciated that the tubes in one group may be staggered relative to those in another group so that water flowing through the gaps in one group will impinge on the tubes in the next group so that no water can flow continuously through all the gaps and avoid contacting some of the tubes. Alternatively, one group of tubes may be rotated about the pipe axis by a selected angle relative to the preceding group so that water will move through the pipe in a somewhat spiraling fashion, also to increase turbulence and improve contact with the tubes.
FIG. 4 further shows that this particular example has been fabricated with end flanges that can mate directly with piping systems using a tri-clamp ferrule system as is familiar in such industrial settings as beverage bottling plants and pharmaceutical production. It will be appreciated that the device may also be conveniently incorporated into clean-in-place systems as are familiar in the aforementioned industrial settings.

### Example

The simplicity of the inventive method was demonstrated by sending the CAD file corresponding to the module in FIG. 4 to a third-party vendor with instructions to make several parts using that vendor's own supply of PLA filament. The vendor selected the process variables for printing based on well established procedures, with no undue experimentation required, and the modules thus fabricated performed well. For convenience in keeping samples separated, the vendor provided parts made from filaments of various colors. Applicants have found that the small amounts of colorants did not have any noticeable effect on performance.

### Example

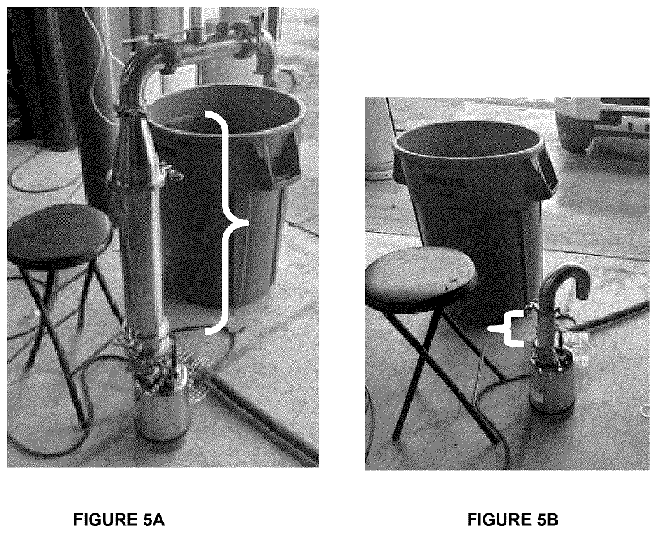

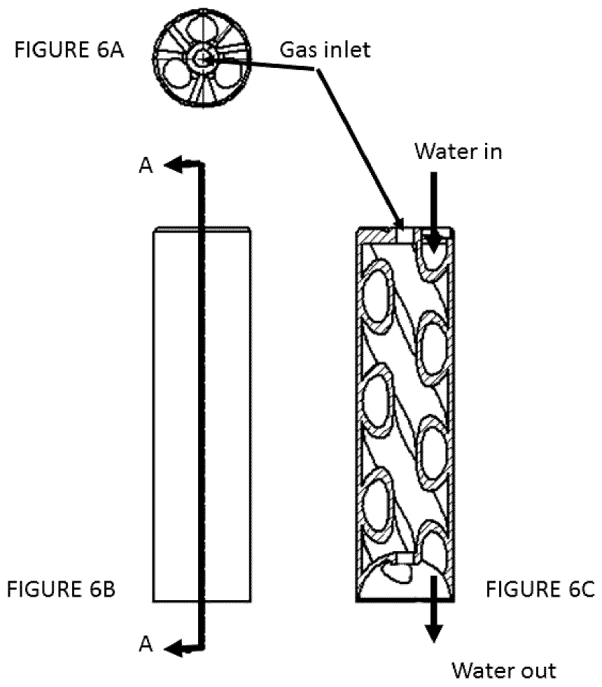

FIG. 5 shows a direct comparison of a conventional infuser module, FIG. 5A (left) and the inventive module from the previous example, FIG. 5B (right). Both modules have equivalent gas infusion capacity. One can see that the inventive module is about ⅙ the size of the conventional module (indicated by brackets). It is also a fraction of the cost and is robust and easily cleaned. Furthermore, the design is easily scaled to virtually any desired size, and easily adapted to any desired tube diameter and interconnection hardware.
The gas exchange module shown in FIG. 5B was sized to produce equivalent oxygenation to that achieved by the conventional module shown in FIG. 5A. Scalability was demonstrated by constructing a module capable of infusing CO₂ at a rate that produces carbonic acid at a pH of around 4 in one pass at a flow rate of about 32 gal/min.

Applicant's use of 3D printing to make the membrane not only eliminates the shortcomings of hollow fiber membranes such as their high cost, large labor input, water flooding, and fragility. It also makes the new membrane inherently scalable in two ways: First, during the design phase, the CAD file may be easily modified, e.g., to increase membrane area or add fluid mixing structures. Second, an existing membrane design may be easily scaled to larger size (limited only by the size of the 3D printer). Third, the membrane shown, e.g., in FIG. 4 forms a compact, robust module so that the skilled artisan may easily configure an infusion unit with many modules ganged in series or parallel to match the capacity of the pumps and meet the specified fluid production rate.

Those skilled in the art will appreciate that processes involving diffusion and aqueous solutions are frequently affected by the presence of boundary layers, which can locally reduce the efficiency of transport. Such boundary layers may be substantially reduced or eliminated by increasing turbulence in the fluid. Applicant observed that the as-formed surface of the membrane, when built using the aforedescribed parameters, has a characteristic texture, which is an artifact of the layerwise build-up of the material. This surface texture characterized by fine grooves might be helpful, either by increasing turbulence, by increasing the effective surface area, or by making the surface effectively more hydrophobic (i.e., increasing the contact angle of a sessile water drop). Applicant further contemplates that one can exploit the unique attributes of the printing process to configure a module with macroscopic internal features deliberately added to increase turbulent flow and improve contact between the fluid and the membrane surface, such as fins or baffles on the inner wall surface.

Although preceding examples described a membrane constructed of polylactic acid (PLA), the skilled artisan will appreciate that many hydrophobic thermoplastics may be suitable for use in the invention, and many are available in appropriate forms to use directly in 3D printing. Some suitable polymers include: polyethylene, polypropylene, nylon, polyethylene terephthalate glycol, and others. Furthermore, some materials can be supplied with fugitive pore-forming additives, which can be removed after the build by dissolving in warm water, for example. One such material is Caverna™ PP Microporous Build Material for Additive Manufacturing (Infinite Material Solutions™, Prescott, WI). Conventionally, these are used for a completely different purpose, viz., to make thin supporting members needed to stabilize the part during build but which will later be removed. Dissolving the additives creates a very porous, weak web that can be easily cleaned away from the denser areas of the structure.

Applicants realized that this principle is also exploited to introduce fine porosity of a known size and distribution, for filtration and other purposes. It could thus be used to potentially further increase the gas permeability of the membrane. In such a case, it will be appreciated that the size, shape, and volume fraction of the additive will preferably be chosen to be at or above the percolation threshold so that continuous gas pathways exist through the membrane wall.

### Example

### Demonstration of the Invention Using Other Polymers.

The following table presents the results of tests infusing oxygen into water using modules constructed of these materials using the same FDM process: Polylactic Acid (PLA), Acrylonitrile Butadiene Styrene (ABS), and Polyethylene Terephthalate Glycol-modified (PETG). The water reservoir contained 32 gal, and the recirculating pump was pumping 32 gal/min so that the water made one complete pass through the exchanger every minute. All test runs were concluded after five minutes. Comparing the results, one can see that PLA and ABS performed almost identically, raising the oxygen content in the water to about four times the normal saturation value. The PETG material effectively transferred oxygen to the water, but at a slower rate; after five minutes (i.e., five passes through the module) the oxygen level was less than in the other two cases, but the number was still rising at a nearly linear rate from 3 minutes to 5 minutes when the run was concluded. It is likely, therefore, that higher oxygen levels would be obtained at longer times.

TABLE 1
Performance of gas membranes made from different polymers.

| | Time, | | | | O₂ flow, |
|---|---|---|---|---|---|
| Material | min | O₂^{a} | TGP^{b} | RES^{c} | L/min |
| PLA | 0 | 86.1 | 101 | 105 | 10 |
| | 1 | 211.6 | 103.5 | 72 | 10 |
| | 2 | 312.4 | 109 | 53 | 10 |
| | 3 | 375.3 | 113.6 | 42 | 10 |
| | 4 | 414.1 | 116 | 36 | 10 |
| | 5 | 437 | 117.5 | 32.5 | 10 |
| ABS | 0 | 96 | 100 | 101 | 10 |
| | 1 | 220 | 103 | 69 | 10 |
| | 2 | 321 | 109 | 50 | 10 |
| | 3 | 380 | 113.7 | 41.1 | 10 |
| | 4 | 418 | 116 | 36 | 10 |
| | 5 | 440 | 118 | 32.4 | 10 |
| PETG | 0 | 94 | 100 | 101 | 10 |
| | 1 | 140 | 101 | 90 | 10 |
| | 2 | 175 | 104 | 84 | 10 |
| | 3 | 207 | 106.9 | 79 | 10 |
| | 4 | 235 | 109 | 75 | 10 |
| | 5 | 261 | 111 | 70 | 10 |

^{a}Dissolved oxygen content, expressed as % of normal saturation level
^{b}Total gas pressure
^{c}Residual gas in water (primarily N₂)

The performance of PETG in this example is especially noteworthy, because conventional thinking has emphasized the use of very hydrophobic materials to construct the membrane. The water contact angle for PETG polymers typically ranges from 64.9° to over 120°, varying based on manufacturing processes, surface treatments, and print settings. One study reported values from 64.9 to 85.6° depending on orientation of the surface layer [see Scherer et al., Bioprocess and Biosystems Engineering (2022) 45:931-41.] A contact angle of 90° is the threshold for a surface to be considered hydrophobic, and PETG can exhibit behaviors on both sides of this threshold. Applicants realized that this surprising result creates completely new opportunities and potential applications for the invention. For example, many situations may include such fluids as oil/water mixtures or dispersions, or water containing various other organics that may be less polar than water by varying degrees. Infusing such mixtures with selected gases could be useful in many situations, and in those cases, a polymer that is less hydrophobic might resist fouling or penetration of organics into the pore spaces.

### Example

The following table lists various polymers in terms of how hydrophobic they are. Hydrophobic properties are often measured by the contact angle formed at the edge of a sessile water drop on the polymer surface, with hydrophobic materials having a contact angle >90° and hydrophilic materials having a contact angle <90°. Guided by this information and the discovery in the previous example that less-hydrophobic polymers can make suitable gas-transfer membranes, the skilled artisan can therefore apply the Invention to many new infusion problems with routine experimentation. Applicants further point out that the speed and convenience of the 3D printing process makes such routine tests very fast and economical compared to evaluating such materials in a traditional hollow fiber membrane, as such hollow fibers will not necessarily be available for all polymers of interest, whereas thick solid fibers suitable for FDM would be much more readily obtained.

TABLE 2
Water contact angles for selected polymer materials.

| | Water |
|---|---|
| | Contact |
| Polymer | Angle, deg |
| Nylon resin (NY) | 74.7 |
| Static Master ESA-9166N [Riken Technos] | |
| Polyacetal (POM) | 76.2 |
| DURACON 270-44 [Polyplastics Co. Ltd.] | |
| Acrylic resin (PMMA) | 79.7 |
| Optimas 7500 [Mitsubishi Gas Chemical Co.] | |
| Cycloolefin copolymer (COC) | 86.0 |
| APEL 5014DP [Mitsui Chemicals] | |
| Acrylonitrile-butadiene-styrene (ABS) | 89.7 |
| Synthetic Resin 191 [Asahi Kasei Chemicals] | |
| Low-density polyethylene (LDPE) | 91.8 |
| NOVATEK-LD LB420M [Nippon Polyethylene Co, Ltd.] | |
| Polystyrene resin (PS) | 95.5 |
| PSJ-Polystyrene 679 [PS Japan] | |
| Polycarbonate resin (PC) | 95.9 |
| Eupilon S 3000R [Mitsubishi Engineering Plastics] | |
| Cycloolefin copolymer (COC) | 96.1 |
| Zonex 690R [Nippon Zeon] | |
| Polybutyrene terephthalate resin (PBT) | 99.4 |
| Novaduran 5510S [Mitsubishi Engineering Co., Ltd.] | |
| Polypropylene resin (PP) | 101.2 |
| NOVATEK-PP MA311 [Nippon Polypro Co., Ltd.] | |

In addition to the filament-based 3D printing process described above, other suitable additive manufacturing processes exist, particularly selective laser sintering (SLS).

### Example

In the SLS process, a powder bed is built up one layer at a time, and a laser selectively bonds powder particles together in such a pattern as to define the solid object as it is built. Exemplary SLS 3D printers include Lisa X and Lisa PRO (Sinterit, Krakow, Poland) and Fuse 1 (Formlabs, Boston, MA). It is understood that the internal cavities in such objects must have an opening for removing unbonded powder from the cavities.
In many of the exemplary designs, there is a single gas inlet to provide pressurized gas to the plenum in the infuser. It will be appreciated that if the device is to be made via the SLS process, it may be desirable to have a gas inlet and a gas outlet, for ease in cleaning the unbonded powder particles from the gas tubes and plenum. The gas outlet may then be capped prior to use.

Many of the foregoing examples describe the infusion of oxygen or CO₂ into water. The skilled artisan will appreciate that there are many applications for gas-permeable membranes for infusion, separation, and other processes, and the relative permeability and separation behavior of many molecular species have been determined. The following table presents data for some of these molecular species using a silicone separator.

TABLE 3
Silicone permeability coefficients for several gaseous species.

| | | Silicone Permeability |
|---|---|---|
| Gas | Formula | Coefficient (Barrer)* |
| Acetone | C₃H₆O | 5860 |
| Ammonia | NH₃ | 5900 |
| Argon | Ar | 600 |
| Benzene | C₆H₆ | 10800 |
| Butane | n-C₄H₁₀ | 9000 |
| Carbon dioxide | CO₂ | 3250 |
| Carbon disulfide | CS₂ | 90000 |
| Carbon monoxide | CO | 340 |
| Ethane | C₂H₆ | 2500 |
| Ethylene | C₂H₄ | 1350 |
| Helium | He | 350 |
| Hexane | n-C₆H₁₄ | 9400 |
| Hydrogen | H₂ | 650 |
| Hydrogen sulfide | H₂S | 10000 |
| Methane | CH₄ | 950 |
| Methanol | CH₃OH | 13900 |
| Nitric oxide | NO | 600 |
| Nitrogen | N₂ | 280 |
| Nitrogen dioxide | NO₂ | 7500 |
| Nitrous oxide | N₂O | 4350 |
| Octane | n-C₈H₁₈ | 8600 |
| Oxygen | O₂ | 600 |
| Ozone | O₃ | 1400 |
| Pentane | n-C₅H₁₂ | 20000 |
| Propane | C₃H₈ | 4100 |
| Sulfur dioxide | SO₂ | 15000 |
| Toluene | C₇H₈ | 9130 |
| Water vapor | H₂O | 36000 |

*1 Barrer = 10⁻¹⁰ cm³ (STP) · cm/cm² · s · cm-Hg

Summary of various aspects of the inventive infusion apparatus.

A gas exchange membrane may comprise:
a rigid, monolithic polymer body defining an internal cavity and an external surface;
a gas inlet integral to the polymer body so that pressurized gas may be introduced into the interior cavity and maintained at a selected pressure therein; and wherein,
the polymer body is sufficiently hydrophobic so that when the body is immersed in water, the gas will diffuse from the internal cavity to the external surface and enter solution in the water.

The polymer body may comprise a thermoplastic material selected from the group consisting of: polyethylene, polypropylene, polylactic acid, nylon, polybutyrene terephthalate, polycarbonate, polystyrene, cycloolefin, acrylonitrile-butadiene-styrene, and polyethylene terephthalate glycol.

The polymer body may be made by an additive manufacturing process, such as fused deposition modeling and selective laser sintering. The polymer material may further contain a fugitive pore-forming additive, which is removed afterward to create a controlled porosity distribution.

The polymer body may further comprise a gas outlet integral therewith. The gas outlet may be open to aid in cleaning the polymer body after fabrication and then closed to maintain the selected gas pressure during operation

The gas may comprise any of the following: air, oxygen, ozone, H₂, CO₂, NH₃, normal alkanes with molecular weights up to n-C₈H₁₈, oxides of nitrogen, methanol, ethanol, and mixtures thereof.

A gas infusion apparatus may comprise:
a rigid, monolithic polymer body defining an internal cavity and an external surface;
a gas inlet integral to the polymer body so that pressurized gas may be introduced into the internal cavity and maintained at a selected pressure therein;
a rigid housing defining a fluid chamber surrounding the polymer body, the housing having a water inlet, and a water outlet, and wherein the gas inlet passes through the housing wall from the polymer body to an external gas connection; and wherein,
the polymer body is sufficiently hydrophobic so that when water is passed through the fluid chamber, the gas will diffuse from the internal cavity to the external surface of the polymer body and enter solution in the water as it passes through the fluid chamber.

The polymer body may further comprise a gas outlet integral therewith and passing through the housing wall to an external gas vent.

The polymer body may comprise a thermoplastic material selected from the group consisting of: polyethylene, polypropylene, polylactic acid, nylon, and polyethylene terephthalate glycol.

The polymer body may be made by an additive manufacturing process, such as fused deposition modeling and selective laser sintering. The polymer material may further contain a fugitive pore-forming additive, which is removed afterward to create a controlled porosity distribution.

The gas may be comprise any of the following: air, oxygen, ozone, H₂, CO₂, NH₃, normal alkanes with molecular weights up to n-C₈H₁₈, oxides of nitrogen, methanol, ethanol, and mixtures thereof.

A method of making a gas infusion apparatus may comprise the steps of:
a) creating a digital solid model defining:
   an outer shell having a water inlet, a water outlet, and a gas inlet,
   an inner hollow body integrally connected to the gas inlet so that a selected gas can be maintained at a selected pressure inside the inner hollow body while the outer shell is filled with water;
b) uploading the digital solid model in a selected file format and resolution to an additive manufacturing system;
c) loading the additive manufacturing system with a feedstock comprising a thermoplastic hydrophobic polymer;
d) building a monolithic polymer body wherein the inner hollow body is sufficiently gas permeable so that when gas is supplied via the gas inlet and water is passed through the outer shell, gas passes through the surface of the hollow body and into the water.

The digital solid model may comprise a file format selected from the group of: STL files, OBJ files, STEP/STP files, and IGES/IGS files. It will be understood that CAD files such as these will go through an intermediate step (“slicing”) which defines the individual layers for printing, with the slice thickness determined by the desired resolution or surface quality of the finished part. Many 3D printers have internal slicing software and accept a CAD file directly; other systems may require the user to do the slicing step with another software package and then input the data into the printer. Any or all of these options are well known to those skilled in the art.

The polymer body may comprise a thermoplastic material selected from the group consisting of: polyethylene, polypropylene, polylactic acid, nylon, and polyethylene terephthalate glycol. The polymer may further comprise a water-soluble pore forming additive. In such a case, the method may further comprise the step of:
e) immersing the formed polymer body in water of a selected temperature for a selected time to dissolve the additive and yield a polymer with a controlled level of porosity.

The outer shell may further comprise an opening with a sealable cover, so that the cover may be temporarily opened to allow access to the interior surfaces for inspection, cleaning, or other purposes. It may further comprise surface features on its interior surface to direct the path of fluid flow and influence turbulent mixing. It may further comprise features on its exterior, such as circumferential ribs for improved mechanical strength and pressure resistance, as well as fluid-impermeable coatings such as epoxy resin, photo-curing resin, and other coating or sealant materials.

A bottling line for beverages may comprise:
a gas infusion module for adding selected gases to a beverage during the bottling process, the gas infusion module comprising:
   a rigid, monolithic polymer body defining an internal cavity and an external surface;
   a gas inlet integral to the polymer body so that pressurized gas may be introduced into the internal cavity and maintained at a selected pressure therein;
   a rigid housing defining a fluid chamber surrounding the polymer body, the housing having a beverage inlet, and a beverage outlet, and wherein the gas inlet passes through the housing wall from the polymer body to an external gas connection; and wherein, the polymer body is sufficiently hydrophobic so that when the beverage is passed through the fluid chamber, the gas will diffuse from the internal cavity to the external surface of the polymer body and enter solution in the beverage as it passes through the fluid chamber.

The beverage may comprise: water, fruit and vegetable juice, coffee, tea, fermented beverages such as beer, ale, and wine, distilled spirits and cocktails, caffeinated energy drinks, and drinks intended for hydration and electrolyte replacement.

The bottling line may bottle the beverage into any conventional containers, including: glass bottles and jars, plastic bottles and jars, heat-sealed aluminum pouches, and metal cans.

The gas may comprise oxygen, hydrogen, or carbon dioxide. Carbon dioxide may be introduced for carbonation, for pH adjustment, or for other purposes.

A clean-in-place system for use in stainless steel process equipment may include a passivation system to provide oxygen-supersaturated water for passivating the stainless steel, wherein the passivation system comprises:
a rigid, monolithic polymer body defining an internal cavity and an external surface;
a gas inlet integral to the polymer body so that pressurized oxygen-containing gas may be introduced into the internal cavity and maintained at a selected pressure therein;
a rigid housing defining a fluid chamber surrounding the polymer body, the housing having a water inlet, and a water outlet, and wherein the gas inlet passes through the housing wall from the polymer body to an external gas connection; and wherein,
the polymer body is sufficiently hydrophobic so that when water is passed through the fluid chamber, the oxygen-containing gas will diffuse from the internal cavity to the external surface of the polymer body and enter solution in the water as it passes through the fluid chamber.

The clean-in-place system may be portable or it may be permanently integrated into the process equipment that is periodically cleaned.

The clean-in-place system may include at least one of the following operations: clean, passivate, and rinse. It may include a control system to follow a selected cleaning cycle. It may further include an oxygen monitor to measure the oxygen level in the water during the passivation cycle.

### Stable Carbonic Acid.

As noted earlier, the inventive apparatus may be used to infuse water with CO₂ to form carbonic acid. Applicants have found that carbonic acid formed in this way can easily be produced to a pH range of 3.8 to 5; in some cases this is achieved in a single pass through the gas exchange module. Applicants have further discovered that the carbonic acid made by this process is remarkably stable over time, even when stored in an open container at ambient temperatures (roughly 65-75° F.) at normal atmospheric pressure (roughly 29.50-30.50 inches of Hg).

### Example

To determine the stability of the inventive carbonic acid, about 40 gal of solution was placed in a plastic barrel with the top open to the air. The barrel was kept in an enclosed shop area to protect from rain and the temperature fluctuated over the course of time as would be expected on a shop floor. The pH was measured hourly at first, then daily, and the results showed that the solution was substantially stable until the test ended after about 40 days. For these purposes, Applicants define such stability as an increase of no more than 10% in the pH value.

### Example

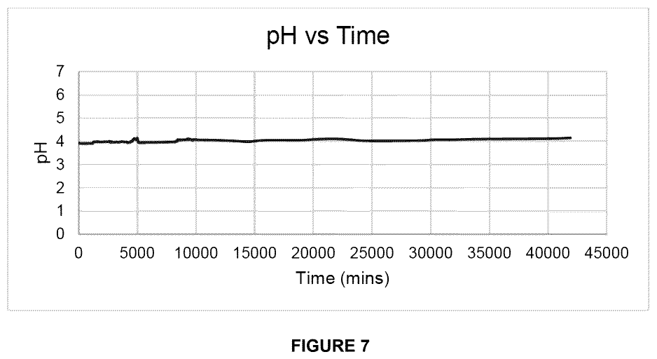

Further stability tests were conducted at an independent research lab. Research Productivity Council (RPC, New Brunswick, Canada) confirmed that the inventive carbonic acid exhibited a stable pH of 3.9 to 4.2, lasting more than 30 days at around 20° C., FIG. 7.

Although the reasons for this remarkable stability are not entirely clear, Applicants postulated that the CO₂ might exist as unusually small bubbles (say, tens to hundreds of nm) or as some form of chemical complexes or clusters that might have distinctive optical properties, such as scattering in the visible range or unique absorption bands in the IR range. However, it will be appreciated that when the analyte is a small amount of gas in water, locating such peaks may be problematic because of the large absorption of the water itself. Applicants studied the optical behavior of a plane polarized beam from a green laser (532 nm) as it passed through samples of distilled water, rain water, and the stable carbonic acid. Applicants observed that the laser beam propagating through the solution was clearly visible when viewed normal to the beam path. The beam was virtually invisible in distilled water. This observation suggested that optical scattering was arising because of interaction of the laser beam with something in the solution.

### Example

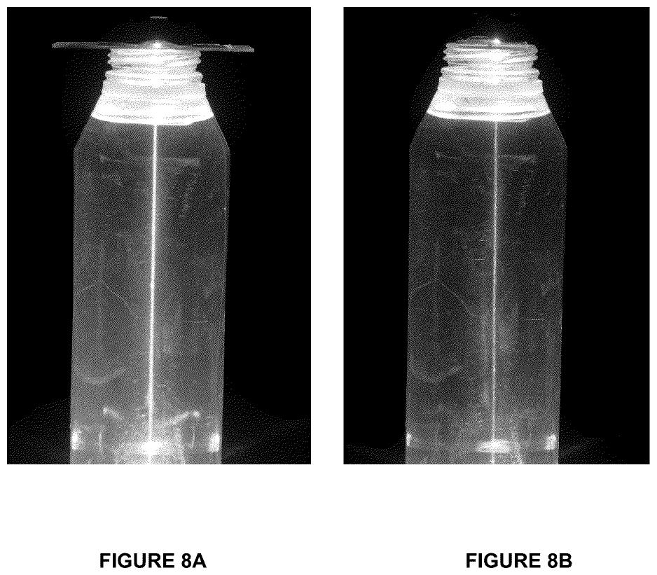

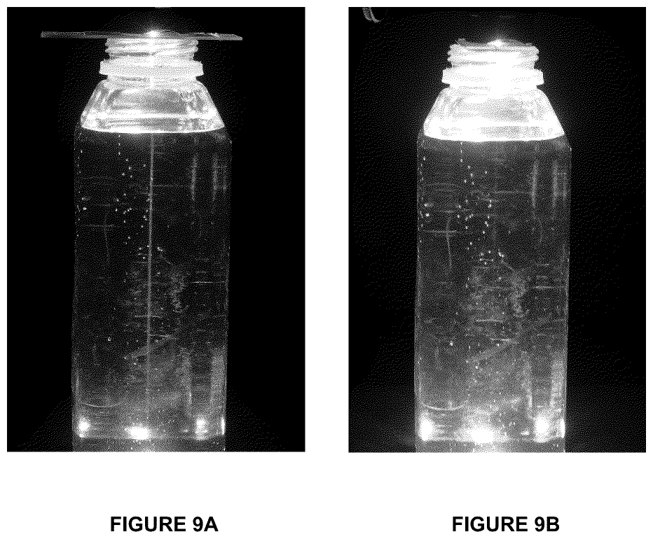

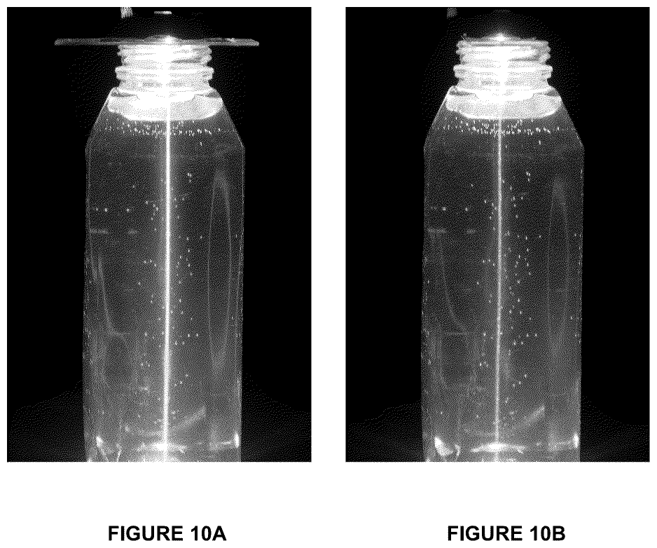

FIGS. 8A-B show images of a laser beam propagating in a carbonic acid solution at pH=4.08. FIG. 8A is viewed in the direction of polarization, and FIG. 8B is viewed normal to the polarization.
FIGS. 9A-B presents images of a laser beam propagating in distilled water solution at pH=7. FIG. 9A is viewed in the direction of polarization, and FIG. 9B is viewed normal to the polarization.
FIGS. 10A-B presents images of a laser beam propagating in rain water at pH=6.49. FIG. 10A is viewed in the direction of polarization, and FIG. 10B is viewed normal to the polarization.

The foregoing example shows that the optical behavior of the green laser beam propagating in these fluids is recognizably distinct from one solution to another. Applicants realized that these differences may be detected by various methods, each method having relative advantages and disadvantages. Such methods include various approaches involving machine vision to quantify differences in the visual appearance as seen by the naked eye in the foregoing photographs.

One useful method, which is potentially simpler than machine vision, involves well-known scattering theories, which relate scattering in the two directions parallel and normal to the plane of polarization to the size of scattering centers and the incident wavelength.

### Example

Three water samples, Table 1, were selected for optical scattering tests using a green laser [Solid Kraft high-powered tactical laser, Class III, <5000 mW, 532±10 nm] and a film-type polarizer [Edmund Optics 26920 Rev 00 UHC linear polarizing film]. It will be appreciated that this green laser is not optimized for precision measurements, but the results are nevertheless indicative that the eye-visible differences can be measured using an optoelectronic approach.

TABLE 4
Water samples for laser scattering studies

| Batch | Description | pH |
|---|---|---|
| 1 | Carbonic acid made by infusion | 4.08 |
| 2 | Distilled water | 7 |
| 3 | Rain water | 6.49 |

Samples shown in FIGS. 8-10 were contained in clear plastic bottles, which were opened for testing. The polarizing film was affixed to a glass slide and placed on the open mouth of the bottle. The laser was placed on top of the film so that the beam propagated downwardly through the liquid, and a camera was positioned to view the beam in the transverse direction so that any scattering of the light would make the beam visible in the photograph. For each sample, two images were acquired; the first was taken from the direction parallel to the polarization (θ₁=0°), and the second was taken from the direction normal to the polarization (θ₂=90°).
It will be appreciated that the scattering behavior of rain water is similar to that of the inventive carbonic acid solution. Applicants attribute this, in part, to the likely suspension of various particulates and other contaminants in the rainwater. Thus, a testing or quality control strategy might include the simultaneous measurement of laser scattering and pH, as the pH of the stable carbonic acid solution (˜4) is significantly lower than that of rain water. This added step will not normally be needed in cases where the carbonic acid is being produced from distilled water which would inherently be free of the impurity particulates found in rain water.

### Example

The experiment above was repeated on fresh carbonic acid (pH 4.02) using a red laser [Class II, <5 mW, 650±10 nm] and a film-type polarizer [Edmund Optics 26920 Rev 00 UHC linear polarizing film]. Overall scattering of the red laser was weaker than that of the green laser, but again the greater intensity of scattering when viewed at 0° compared to 90° was clearly visible to the naked eye.

### Example

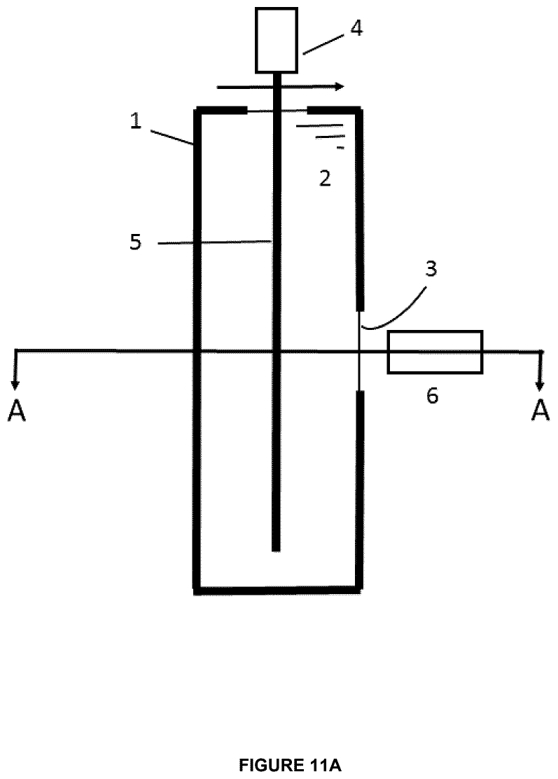

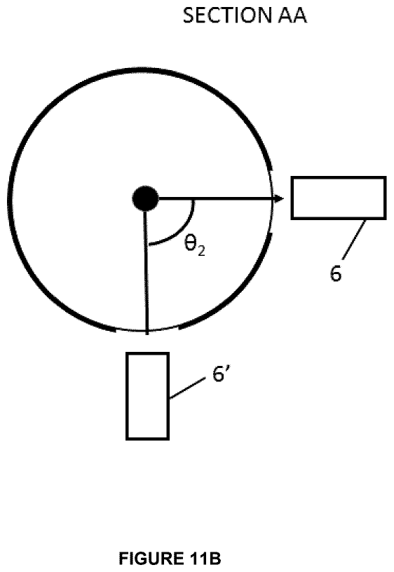

FIG. 11A-B is a schematic diagram of a sensor module in accordance with one aspect of the invention. FIG. 11A is an elevation view in cross section and FIG. 11B is a cross section through A-A, and the arrow indicates the direction of polarization of the laser beam. Container 1 holds fluid 2 to be analyzed; the fluid may be static (as shown) or container 1 may be provided with inlet and outlet lines (not shown) so that the fluid may pass continuously through the module as a process control step. Laser 4 provides a beam 5 passing down through fluid 2. Beam 5 is polarized as indicated by the arrow. The laser may be configured to provide polarized output; alternatively, a polarizing filter may be placed in the beam path as is known in the art. Container 1 is provided with several windows 3 to allow the input beam to enter and allow scattered light to exit to photodetectors 6, 6′. One photodetector 6 is oriented to capture light scattered at θ₁=0, i.e., parallel to the plane of polarization; the second detector 6′ is oriented at some nonzero angle to the polarization (preferably θ₂=90°) as shown in FIG. 11B.

Those skilled in the art will recognize that light intensity can be detected by using:
1. Photoresistors (Light-Dependent Resistors—LDRs): These sensors detect light intensity and change their resistance based on the amount of light. They are commonly used in automatic street light systems and other applications where light intensity is a variable factor.
2. Photodiodes: These sensors convert light into an electric current. They are sensitive and can measure light intensity accurately. Photodiodes are often used in automation systems and industrial controls.
3. Phototransistors: Similar to photodiodes but with an additional amplification stage, phototransistors offer high sensitivity and current gain, making them suitable for low-light applications.
4. Photovoltaic Cells: These cells convert light energy directly into electrical energy through the photovoltaic effect. They are commonly used in solar energy applications.

### Example

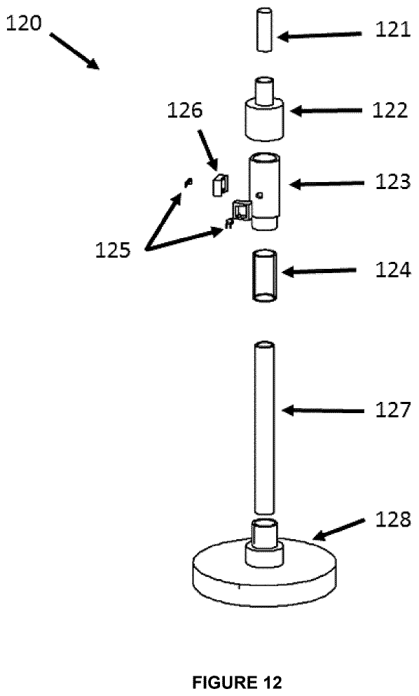

FIG. 12 illustrates an exploded view of a prototype sensing device in accordance with the physical principles shown in FIG. 11. Photoresistors (LDR) were used in this initial test because of their simplicity to implement, but because they are used in a voltage divider configuration, resistance variations due to manufacturing tolerances, contamination, and temperature influences can cause variations in the light meter output that are not due to laser reflection/refraction. Thus, for a more precisely calibrated sensor, particularly one used in process control and monitoring as the carbonic acid is produced, phototransistors with signal conditioning operation amplifiers would be a preferred long-term solution. Furthermore, the prototype system was constructed using individual mechanical components that were 3D printed. It will be appreciated that more precise machined parts will be preferred for a well-calibrated sensor for production control.
FIG. 12 illustrates a sensor 120 configured for batch analysis. The carbonic acid solution is held in clear tube 124. Light from laser 121, which is inserted into laser holder 122, propagates downward through tube 124. Detector holder 123, surrounds tube 124 and holds two photoresistors 125 contained in potting boxes 126. As shown, the two photoresistors are preferably 90° apart, and one of the two is aligned with the plane of polarization and the other is normal thereto. Extension tube 127, which connects to base support 128, is intended to function as a crude beam dump to minimize internal reflections that would add noise to the signals in the two photodetectors.
The measurement circuit consists of two light dependent resistors (LDR), type 5528 [Seeed Technology Co., Ltd., Shenzhen, China], 5 mm diameter. This LDR has a resistance range of approximately 10-20 KΩ in bright light and >1 MΩ in darkness.
Each LDR is wired with a 10 KΩ (±1%) metal film fixed resistor in a voltage divider configuration.
A potential of 5 VDC feeds into each voltage divider circuit, with the outputs going to the first two analog inputs on a microcontroller board based on the ATmega328P.
A 16 character, two line liquid crystal display (LCD) is connected to the I²C communication channel of the microcontroller. Data values from the two optical detectors are displayed on the two lines of the LCD as percentages of the range of the analog inputs (a value between 0 and 1023).

### Example

### Test Results

The device described above was filled with carbonic acid solution (pH 4.02) and the sensor outputs were recorded. The detected signal from the sensor oriented parallel to the polarization (0°) was about twice the signal from the sensor oriented normal to the polarization (90°).

### Example

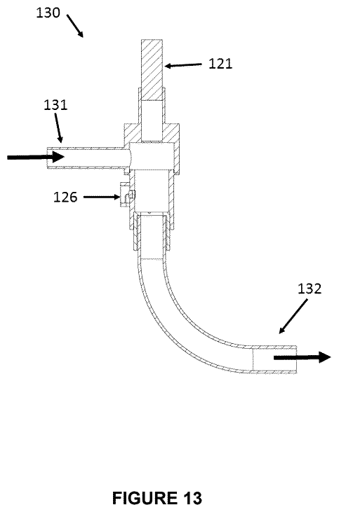

FIG. 13 illustrates schematically in cross section a sensor 130 configured to measure the scattering in a fluid continuously flowing through the system (arrows) via inlet 131 and outlet 132. The optical sensor and potting box 126 (one of two is shown in FIG. 13) are the same as in FIG. 12.

### Example

The inventive sensing approach lends itself to a feedback control system in which the laser scattering sensor is placed in the outflow stream of the gas infuser, and if the ratio of intensities at θ₁=0° and θ₂=90° falls below a predefined ratio, an alarm state might be initiated. Alternatively, a fluid control system including pumps, valves, etc., may receive inputs from the sensor so that the flow rate can be reduced or increased until the ratio is back within the target range.

In the physical description of optical scattering phenomena generally, the strong wavelength dependence of the scattering (˜λ⁻⁴) means that shorter (blue) wavelengths are scattered more strongly than longer (red) wavelengths. Furthermore, scattering is influenced by the size of the particle (or gas bubble), r, relative to the wavelength of the light, λ. It follows from this that by observing the visual intensity of the laser beam at two different angles relative to the polarization, and simultaneously performing the same analysis at two different laser wavelengths, further information may be derived regarding the actual state and physical characteristics of the CO₂ in the carbonic acid fluids of interest.

Those skilled in the art will appreciate that laser light sources are available that operate on various selected wavelengths, and that measurements such as those described in the preceding example may be done at more than one wavelength in parallel and/or simultaneously.

### Example

A sensor module may be provided with two lasers producing two different colors to analyze fluid samples. Four photodetectors may be provided, one pair having a first color filter corresponding to the first laser beam and the second pair having a second color filter corresponding to the second laser beam. Each pair of detectors would be arranged at different angles θ₁, θ₂, and θ₁′, θ₂′ respectively relative to the polarization (preferably θ₁, θ₁′=0 and θ₂, θ₂′=90°) so that the relative scattering ratios of the two colors may be used to quantify the characteristics of the scattering centers (e.g., the effective size of microscopic gas bubbles) or some other measure of quality of the fluid being produced.
A sensor module may be provided with a white light laser. Four photodetectors may be provided, one pair having a first color filter and the second pair having a second color filter. Each pair of detectors would be arranged at different angles relative to the polarization (preferably 0 and 90°) so that the relative scattering ratios of the two colors may be used to quantify the characteristics of the scattering centers (e.g., the effective size of microscopic gas bubbles).

An additional process control strategy can involve placing a pH meter in the outlet line, so that the control system may rely on the pH readings and a preset control or target pH level. As the system runs, if the pH falls outside the target range, the water flow may be adjusted upward or downward to restore the product to the target pH value. Many suitable pH sensors are familiar in the art and readily available from many sources; the skilled artisan may therefore select a pH meter or sensor having a desired form factor, range, precision, etc. for any particular control system and operating strategy.

The control system may perform additional functions as are desirable or appropriate in various uses. For example, the system may contain a flow meter on the output side that records total flow accumulated over time. So a user might rent or lease a system and be billed based on a charge or royalty per gallon of carbonic acid produced; in-line sensors (optical device or PH meter) could verify in real time that the fluid dispensed was within specification.

Infusion of More than One Gas into a Solution.

It will be appreciated that the invention may be used to make infusions containing more than one gas. Examples include formulating beverages infused with both CO₂ and H₂ or producing skin treatment solutions, creams, or gels infused with CO₂ and O₂. Applicants have found that various combinations can be produced and, surprisingly, the results can depend on the order in which the different species are infused, as described in the following examples.

### Example

An infusion module made from PLA material was connected to a 32-gal barrel of rain water (starting pH 5.81 and oxygen at 92.5% of saturation) and a recirculating water pump running at 32 gal/min so that the entire volume of water was turned over once per minute. The water was first infused with oxygen at 10 L/min. After 5 min O₂ was 366% of saturation and pH was 6.51.
The oxygen-infused water was then infused with CO₂ at 10 L/min for five minutes, after which O₂ was 253% of saturation and pH was 4.32. This test demonstrates that 02-infused carbonic acid can be produced while retaining a favorable pH.
The O₂-infused carbonic acid was then infused with H₂ at 10 L/min and after one minute (i.e., one pass through the gas exchange module) oxygen was reduced to about 121% of saturation and hydrogen was at 1 ppm.

### Example

Starting with fresh rain water (pH 5.91 and oxygen saturation at 93% of saturation) CO₂ infusion was run for 5 min. The resulting carbonic acid had pH=4.29 and oxygen at 72% of saturation.
The carbonic acid was then infused with oxygen for five minutes at 10 L/min and this infusion brought the oxygen to 291% of saturation at a pH=4.39. These results suggest that of maximum oxygen is desired in the carbonic acid, then it is preferable to infuse the water first with CO₂ and then with O₂, rather than vice versa.

### Applications of Stable Carbonic Acid.

### Firefighting and Fire Suppression.

The addition of carbonic acid to water and firefighting foams creates an added fire suppressing effect via the decomposition of carbonic acid into CO₂ and water. The inventive stable carbonic acid solution is particularly suitable for these applications, and the inventive gas infusion module may be conveniently incorporated into numerous firefighting systems as described in the following examples.

### Example

Wildfires and forest fires often occur in remote areas where access to water may be severely limited. In extreme cases, specially designed aircraft may be used to scoop water from a lake and then drop it on the fire. For such scenarios, the aircraft could be modified to include: a recirculating water pump, a gas infusion module, and a tank of compressed CO₂ or more preferably, a tank of liquid CO₂. The pump and infusion module would preferably be sized so that the water in the tank can be fully infused while in flight between the water source and the fire. The on-board CO₂ supply tank will preferably be configured such that when the CO₂ needs replenishing, the aircraft can land and be quickly refilled with CO₂ and then resume firefighting operations. To this end, the fill port for the CO₂ tank will preferably be on the outside of the aircraft for rapid access and filling by the ground crew.

### Example

Brush fires or building fires that occur in some urban or semi-urban settings such as the urban wildlands of Los Angeles County might or might not have access to city water (i.e., fire hydrants). Pump trucks will either draw hydrant water directly or draw water from separate tank trucks that periodically return to city water supplies for replenishment. The inventive gas infusion apparatus may therefore be configured in several ways to provide the maximum flexibility for particular missions.
A CO₂ tank, recirculating pump, and infusion module may be added directly to the pump truck, or as a trailer tethered thereto, in order to add carbonic acid to the water just before release into the fire hoses.
A CO₂ tank, recirculating pump, and infusion module may be added to the water tank truck so that the water can be infused either while in route to the fire scene, or directly as it is supplied to the pump truck.
Alternatively, a separate “infuser” truck may contain a large CO₂ supply, an infusion module, and water inlet and outlet connections. At the fire scene, the water inlet would be connected to a fire hydrant or a water tank truck, and the water outlet would be connected to the pump truck. In this configuration the pump truck would generally provide the pumping power to draw the water through the infuser.

Those skilled in the art will readily appreciate that the concepts described in the preceding examples may be extended to other firefighting systems as are known in the art. For example, water dispensing systems may include nozzles, hoses, monitors, etc. In addition to the stable carbonic acid, the water may include various additives, fire suppressants, foams, etc. The water dispensing systems may be familiar municipal fire trucks or may be any nozzle mounted on any kind of vehicle. Nozzles and monitors may be manually operated or may have any desired motion controls or automation.

Those skilled in the art will appreciate that the inventive carbonic acid may be further combined with additives such as foam formers, surfactants, and other chemicals as are familiar in the art. Examples include fire suppressants [e.g., PHOS-CHEK® and FIRE-TROL® products from Perimeter Solutions, Clayton, MO], firefighting foams [e.g., AUXQUIMIA® products from Perimeter Solutions, Clayton, MO] and water enhancers (gels) [e.g., Eco-Gel™ products from FireRein, Napanee, Ontario, Canada].

### Beverages.

Beverages may be infused with various gases for various purposes; the gas may be oxygen, hydrogen, or CO₂. The inventive infusion apparatus may be incorporated directly into an existing bottling or canning line in order to make infused water directly as the desired beverage is formulated and bottled. Alternatively, the infusion system may be a separate line that produces infused water for storage in a holding tank, from which it may be piped to various bottling lines as needed.

Alcoholic beverages (beer, ale, wine, and distilled spirits) may be infused with oxygen, hydrogen, or CO₂ prior to placement in aging vats or barrels in order to influence the progress of oxidation or other chemical effects of the aging process.

### Hydrometallurgy.

Heap leaching is a familiar hydrometallurgical process used extensively in recovering a number of metals, in particular Cu and Au. In practice, a leach pad is constructed with a bottom slope of 5° or so, leading to a ditch running along the length of the pad. The surface of the pad is typically covered with a water-impermeable material, such as plastic sheeting. The leach pad is typically large, e.g., 100 m in the lateral dimensions. Crushed ore is piled on the pad to a selected depth; the crushed ore is typically the consistency of fine gravel because the bed must optimize the competing factors of surface area and permeability to the leaching solution. An array of small shower heads are arranged along the upper surface of the ore pile and leaching solution is periodically sprayed on the ore. The leaching solution then slowly trickles down through the bed and eventually a metal-rich solution collects in the ditch. In the case of copper, for example, the leaching solution is typically sulfuric acid at a strength of 100 g/L, more or less depending on the mineral makeup of the ore material.

### Example

Testing procedures have long been used in the industry to determine the optimal conditions for leaching ore from a particular mine site. Typically a 12-in diameter column is set up and filled with crushed ore (maximum particle size ⅜, ½, or ¾ in). Different leach solutions and flow rates may be tested over extended periods and the metal concentration and pH of the pregnant leach solution monitored. Using such a test column, stable carbonic acid may be added as a supplement to the sulfuric acid to determine if the amount of sulfuric acid may be reduced when processing a particular ore material.
When it is determined that carbonic acid has a beneficial effect in a particular ore, an infusion station would preferably be set up on site and connected to the existing water supply. CO₂ would then be delivered, either in liquid form or as tanks of compressed gas. The amount and rate of CO₂ consumption is easily calculated based on the column tests so that an appropriate delivery schedule for CO₂ shipments can be set up.

In situ leaching is an economically important process for recovering certain metals, particularly uranium, from deeply buried sedimentary strata. It is used when the ore layer is somewhat permeable to fluids, is bounded above and below by relatively impermeable strata, and extends continuously over a relatively large area. The depth and geometry of such deposits often makes conventional underground or surface mining methods impractical or impossible.

The in situ leaching process may be described in simple terms by the steps of:
a) drilling an injection well and at least one extraction well into a permeable layer of a selected metal ore (typically there are many extraction wells for each injection well in an ISL operating field);
b) forming a leaching solution whose composition will depend on the particular mineralogy present (for sandstone-hosted uranium deposits, both acidic and alkaline chemistries have been used commercially);
c) injecting the leaching solution into the injection well to dissolve and mobilize the targeted metal species; and,
d) pumping the metal solution from the extraction wells and piping it to a processing facility in order to recover the selected metal.

Although the reactivity of carbonic acid at a pH of 4 to 5 is likely not sufficient to effectively mobilize U from carnotite ores, for example, Applicants realized that the stable carbonic acid of this invention may be used as an adjunct or supplement to existing ISL protocols. The benefits of doing this would include reducing consumption of other acids or reagents and productively using waste CO₂ from fossil fuel consumption. Furthermore, a relatively compact infusion station may be set up at the ISL site so that only CO₂ needs to be trucked in as compressed gas or liquid, or in some cases available via pipeline, and then infused directly into the water before making up the final injection solution and pumping into the injection well.

The skilled artisan can, therefore, through routine experimentation using samples of the local ore, compare various leaching chemistries to determine the effect of varying amounts of carbonic acid on leaching efficiency.

To apply carbonic acid to the recovery of target metals in a tailings pond, an infusion system may be set up at the edge of the pond and provided with a supply of CO₂. The system would pump water from the pond, pass it through the membrane exchanger, and discharge it back into the pond. It will be appreciated that the position of inlet and outlet points within the pond may be selected to optimize the infusion efficiency and carbonic acid retention. For instance, the inlet water may be taken from water near the surface and the infused water may be injected at some greater depth at a different location.

### Environmental Remediation.

Ash and waste ponds, particularly fly ash ponds, tend to be highly alkaline. Injection of carbonic acid to the water could serve three purposes: first, reducing the pH of the water; second, forming carbonate minerals to cement the sediment into a more solid form and thereby reduce leaching of undesired species such as heavy metals into the water; and third, to sequester CO₂.

### Inorganic Synthesis.

The inventive stable carbonic acid is a promising new tool for inorganic synthesis, particularly in the field of so-called templated growth of new inorganic crystal structures.

Templated growth in zeolites refers to the process where organic or inorganic molecules (templates) direct the formation of zeolite structures during synthesis. These templates control the size, shape, and connectivity of the zeolite's pores, leading to materials with tailored properties for various applications. Zeolites are crystalline aluminosilicate materials with a framework structure containing micropores (i.e., small cavities and channels). These materials are widely used as catalysts, adsorbents, and ion exchangers in various industrial processes because the size, shape, and chemical properties of the pores can be engineered to create very specific properties. The templates, in this case, tend to be soluble clusters or complexes that act as structure-directing agents, guiding the arrangement of the structural building blocks (SiO₄ and AlO₄ tetrahedra) into specific crystalline structures. Templating complexes can also occupy and thereby define the space within the growing zeolite framework, influencing the size and shape of the resulting pores. In some cases, templates can also help balance the charge within the zeolite framework, which is crucial for maintaining the overall structure. Templates may be organic or inorganic: organic templates are often used and include molecules such as quaternary ammonium cations, amines, and alcohols; inorganic templates include alkali cations like Na⁺, K⁺, and Cs⁺. The growth process is typically done in a hydrothermal environment, in which the zeolite precursors are mixed with templates and water, and then heated under pressure in an autoclave. The template molecules influence the crystallization process and the resulting zeolite structure.

There are several benefits of templated growth: Templated synthesis allows for the precise control of pore size and shape, which is crucial for optimizing the performance of zeolites in various applications. Templated growth can also create zeolites with enhanced properties, such as increased surface area, improved diffusion of molecules, and tailored catalytic activity. Templated synthesis can be used to create novel zeolite structures with unique properties.

Those skilled in the art will therefore appreciate that the inventive carbonic acid affords the opportunity to add another dimension to the process of synthesizing zeolites and other useful inorganic phases, either by allowing the chemist to create different stable complexes in the synthesis reactor or by adding nanoscale gas bubbles to create mesoporous areas in a forming gel or crystalline structure.

### Gas-Infused Compositions for Skin Care, Wound Care, and Cosmetics.

There is significant interest at present in the usefulness of various dissolved gases, and carbonic acid solutions, in topical skin treatments, burn treatments, cosmetics, etc. [see Seidel and Moy, “Effect of Carbon Dioxide Facial Therapy on Skin Oxygenation,” J. Drugs Dermatol. 14(9): 976-980.] Topical treatments may comprise a number of modalities and form factors. Salves, creams, and ointments may include various individual components, such as colorants (in the case of cosmetics), thickeners or other rheology control agents, oils and emollients, surfactants, active drugs, fragrances, and inert ingredients. Topical application may also rely on prepackaged structures such as woven or nonwoven pads, which might further contain a supported layer of cross-linked hydrogel material as is familiar in prepackaged burn and wound dressings.

Applicants conducted a number of experiments to determine if the inventive gas infusion process might be adapted to these treatment materials and methods, as described in the examples that follow.

### Example

A stable carbonic acid solution was prepared in accordance with the Invention to pH=4.1. The solution was then mixed with a thickening agent (Clear DysphagiAide® Beverage and Food Thickener, ERB Unlimited LLC, Savannah, GA). The resulting mixture formed a gel with a nectar-like consistency suitable for topical application. The gel had a pH of about 4.4. Applicants attribute some of the loss of pH to the vigorous stirring needed to disperse the thickening agent into the solution; however the final product was still well within useful the range of carbonic acid.

### Example

The ability to load a cross-linked hydrogel was tested using “water beads” that are used for various applications, such as supporting cut flowers in a vase, children's toys, etc. The beads are composed of a water-absorbing superabsorbent polymer (SAP, also known as slush powder in dry form) such as a polyacrylamide (frequently sodium polyacrylate).
As-received (dehydrated) polyacrylamide gel beads were immersed in the inventive carbonic acid solution, and they swelled normally as they became hydrated with the carbonic acid solution. When these beads were removed from the solution and placed in water, the pH of the water gradually fell as the carbonic acid diffused from the beads to equilibrate with the surrounding water. This demonstrates that the carbonic acid is carried into the hydrogel as it absorbs water and remains chemically active in the hydrogel body.
This example shows that a supported hydrogel pad in a prepackaged dressing for burn or wound care may be conveniently loaded with carbonic acid. Furthermore, as Applicants have demonstrated in several examples above, the water in the gel may be further infused with oxygen in order to provide additional benefits to the patient.

### Example

The same cross-linked SAP is available in crushed or powdered form [Soil Moist™ granules, JRM Chemical, Inc., Cleveland, OH]. The granular material was mixed with stable carbonic acid solution (pH 4.09) and it hydrated normally, as did the gel beads in the previous example.

Those skilled in the art will appreciate that with routine experimentation the inventive carbonic acid solution may therefore be combined with rheology-controlling agents including thickeners such as guar gum, xanthan gum, alginate, methyl cellulose, cornstarch, inulin, polyethylene glycol, etc., and surfactants to make a topical cream or paste of any desired consistency that will allow the carbonic acid to be retained conveniently in the treatment area. Sheets of cross-linked hydrogel, either alone or supported by a fabric or other flexible material, as are well known in the field of burn dressings, may be hydrated with the carbonic acid solution and wrapped on the damaged skin. The hydrating solution may be further infused with oxygen or hydrogen as disclosed above.

It will be appreciated that the inventive gels may be further modified by adding various pharmacologically active agents, which may include drugs, antibiotics, antifungal agents, vitamins, amino acids, herbal extracts, etc., as are known in the art of topical skin treatments.

### Gas-Infused Compositions for Oral Administration.

A growing body of work (3000+ peer reviewed studies) suggests that H₂-infused water confers a number of possible health and wellness benefits. It is a selective antioxidant that can react with such problematic free radicals as hydroxyl radicals and peroxy-nitrite while preserving beneficial immune signaling. It can reduce oxidative stress markers throughout the body. It can reduce inflammation through IL-6 and TNF-α down regulation.

H₂-enriched water is available to consumers in familiar single-serving soft drink cans [e.g., Elevate™ premium hydrogen-infused water, Elevate Beverages, Westlake Village, CA; H2ForLife, Faith Springs, LLC, Blue Ridge, GA]. Alternatively, home generators may be used to make the infused water for immediate consumption. In either case, the infused water must be consumed quickly because the hydrogen is rapidly lost to atmosphere. Elevate Beverages recommends drinking the entire can in five minutes; H2ForLife recommends drinking within fifteen minutes of opening.

Applicants have found, surprisingly, that H₂-infused water made according to the invention has dramatically greater stability, e.g., five days versus minutes to hours in conventionally infused products, as described in the following example.

### Example

The module illustrated in FIGS. 4A-D, produced by 3D printing using PLA material, was operated with a 32 gal/min recirculating water pump and supplied with hydrogen gas metered to deliver 5 L/min at 20 psi and placed into an open top 32 gallon drum filled with distilled water. The gas supply and pump were operated for 2 minutes and 30 seconds. At the end of the test, the fluid was measured immediately and then periodically for 7 days. The results are shown in Table 5. The test was then repeated and the results are shown in Table 6.

TABLE 5
Hydrogen concentration versus time stored in open barrel.

| | H₂ concentration | Oxygen Reduction |
|---|---|---|
| Time (days) | (ppm) | Potential (mV) |
| 0 | 2.196 | −733 |
| 1 | 1.782 | −596 |
| 5 | 1.11 | −400 |
| 6 | 1.116 | −386 |
| 7 | 0.906 | −256 |

TABLE 6
Hydrogen concentration versus time stored in open barrel.

| | H₂ concentration | Oxygen Reduction |
|---|---|---|
| Time (days) | (ppm) | Potential (mV) |
| 0 | 2.124 | −727 |
| 1 | 1.752 | −585 |
| 2 | 1.644 | −552 |
| 5 | 1.242 | −418 |
| 6 | 0.768 | −259 |
| 7 | 0.387 | −129 |

Persons of ordinary skill will readily appreciate that the fluid so produced is highly enriched in hydrogen and remains so for an extended period of time. It will be further appreciated that the stability of the fluid has clear advantages. Among other things, the stability simplifies packaging: since the fluid does not immediately off-gas hydrogen in significant quantities after preparation at atmospheric pressures, it can, for example, be dispensed into cans in conventional canning lines. Stability also increases consumer acceptance as a packaged (canned) beverage. The entire contents of the package need not be immediately consumed (within minutes after opening) and the fluid can be mixed with ice, poured, and even shaken without unreasonable loss of hydrogen.

Because of the excellent stability of the inventive infusion, H₂-infused water was combined with a commercial food-grade thickener to make a consumable gel product, as described in the following example.

### Example

H₂ infused water (1.7 ppm H₂) was prepared as described above. A xanthan gum-based commercial powder [Clear DysphagiAide® Beverage and Food Thickener, ERB Unlimited LLC, Savannah, GA] was dispensed into a dry glass using the included measuring scoop. About ½ cup of H₂-infused water was added to the dry thickener while stirring briskly for about 30 seconds to obtain a nectar-like consistency. The mixture was allowed to set for 4 h and then the hydrogen level was measured daily, Table 7.

TABLE 7
Hydrogen concentration versus time in xanthan gum based gel.

| | H₂ Concentration | Temperature |
|---|---|---|
| Time (days) | (ppm) | (° C.) |
| 1 | 1.524 | 22.9 |
| 2 | 1.539 | 22.7 |
| 5 | 1.467 | 22.7 |
| 6 | 1.428 | 22.3 |
| 7 | 1.472 | 22.7 |
| 8 | 1.44 | 22.0 |

This example demonstrates that the inventive H₂-infused water is stable when mixed with a conventional food-grade thickener.

It will be appreciated that various ingredients may be added to orally-administered formulations, such as flavors, colors, etc. Some of these additives might react with hydrogen or otherwise render it less potent through extended storage. Thus, for added shelf life, a single-dose package might contain two separate cells: an aluminum foil pouch containing the H₂-infused gel, and an aluminum foil or plastic pouch containing any other liquid ingredients. The user will then open both pouches and dispense the two components directly into the mouth for consumption.

We claim:

1. A carbonic acid solution comprising:
an aqueous infusion of CO₂ having a pH≤5 and characterized in that said pH is substantially stable for at least 30 days in an open container at ambient temperature and normal atmospheric pressure.

2. The carbonic acid solution of claim 1 further characterized in that the scattered intensity of a polarized laser beam passing therethrough is at least 2 times greater in the direction parallel to said polarization than the scattered intensity in the direction normal to the plane of polarization.

3. The carbonic acid solution of claim 2 wherein said laser beam comprises green light at a wavelength of 532±10 nm.

4. The carbonic acid solution of claim 2 wherein said laser beam comprises red light at a wavelength of 650±10 nm.

5. The carbonic acid solution of claim 1 further infused with a selected level of oxygen.

6. The carbonic acid solution of claim 1 further infused with a selected level of hydrogen.

7. The carbonic acid solution of claim 1 further comprising a rheology-controlling additive selected from the group consisting of: surfactants, thickeners, guar gum, xanthan gum, alginate, methyl cellulose, cornstarch, inulin, and polyethylene glycol.

8. The carbonic acid solution of claim 7 further comprising an additive selected from the group consisting of: pharmacologically active agents, drugs, antibiotics, antifungal agents, vitamins, amino acids, and herbal extracts.

9. The carbonic acid solution of claim 1 further comprising a super-absorbent polymer.

10. The carbonic acid solution of claim 9 wherein said super-absorbent polymer comprises individual hydrated granules.

11. The carbonic acid solution of claim 9 wherein said super-absorbent polymer comprises a continuous hydrated sheet.

12. A method of making a stable carbonic acid solution comprising the steps of:
a) providing a gas infusion module comprising a monolithic hydrophobic gas-exchange membrane including a water inlet, a gas inlet, and a water outlet;
b) providing a source of pressurized water from a selected supply into said water inlet;
c) providing a source of CO₂ gas into said gas inlet;
d) flowing said water through said gas infusion module while supplying CO₂ to said gas inlet to form a carbonic acid solution of a selected composition and pH.

13. The method of claim 12 comprising the further steps of:
e) providing a monitoring device downstream from said water outlet to monitor at least one property of said stable carbonic acid solution; and,
f) providing a control system to adjust the flow of said inlet water based on feedback from said monitoring device.

14. The method of claim 12 further comprising the step of:
g) providing a source of a second gas and infusing said second gas into said solution in accordance with steps (c) and (d).

15. The method of claim 14 wherein said second gas is oxygen and steps (c) and (d) are carried out first with CO₂ and then with oxygen to form a solution having a pH≤5 and an oxygen content of at least 200% of saturation.

16. The method of claim 14 wherein said second gas is hydrogen and steps (c) and (d) are carried out first with CO₂ and then with hydrogen to form a solution having a pH≤5 and a hydrogen content of at least 0.5 ppm.

17. The method of claim 14 further comprising the step of:
h) providing a source of a third gas and infusing said third gas into said solution in accordance with steps (c) and (d).

18. The method of claim 17 wherein said first gas is CO₂, said second gas is oxygen, and said third gas is hydrogen and steps (c) and (d) are carried out first with CO₂ and then with oxygen, and then with hydrogen to form a solution having a pH≤5, an oxygen content of at least 120% of saturation and a hydrogen content of at least 0.5 ppm.

19. A hydrogen-infused aqueous solution containing 0.5 to 1.5 ppm H₂ and characterized in that said hydrogen content is substantially stable for at least five days in an open container at ambient temperature and normal atmospheric pressure.

20. A method of making a hydrogen-infused aqueous solution comprising the steps of:
a) providing a gas infusion module comprising a monolithic hydrophobic gas-exchange membrane including a water inlet, a gas inlet, and a water outlet;
b) providing a source of pressurized water from a selected supply into said water inlet;
c) providing a source of H₂ gas into said gas inlet;
d) flowing said water through said gas infusion module while supplying H₂ to said gas inlet to form a solution having a hydrogen concentration of 0.5 to 1.5 ppm H₂.
